(19) United States
(12) Patent Application Publication
(10) Pub. No.: US 2019/0048425 A1
(43) Pub. Date: Feb. 14, 2019

(54) TUMOR DETECTION AND MONITORING

(71) Applicant: GENETICS RESEARCH, LLC, D/B/A ZS GENETICS, INC., Wakefield, MA (US)

(72) Inventor: Anthony P. Shuber, Northbridge, MA (US)

(21) Appl. No.: 16/159,116

(22) Filed: Oct. 12, 2018

Related U.S. Application Data
(63) Continuation-in-part of application No. 16/018,926, filed on Jun. 26, 2018.
(60) Provisional application No. 62/672,217, filed on May 16, 2018.
(60) Provisional application No. 62/526,091, filed on Jun. 28, 2017.

(51) Int. Cl. C12Q 1/6886

(52) U.S. Cl. CPC C12Q 1/6886; C12Q 2600/112; C12Q 2600/156

(57) ABSTRACT

The invention provides methods for monitoring cancer reoccurrence in an individual. Methods of the invention include identifying passenger mutations specific to an individual and detecting the passenger mutations by capturing target nucleic acid directly from bodily fluid samples, without the need for certain complex sample preparation steps, using Cas endonuclease to bind to the target nucleic acid sequences. The detection of passenger mutations specific to an individual provides the ability to monitor the reoccurrence of cancer in an individual. The presence of passenger mutations in a sample obtained subsequent a treatment, is indicative of the reoccurrence of cancer. Methods of the invention provide Cas proteins, along with their sequence-specific guide RNAs, may be introduced directly into the sample, where the Cas proteins bind to ends of a target nucleic acid. The target nucleic acid is thus isolated or enriched in a sequence-specific manner. The target nucleic acid may then be subject to any suitable detection or analysis assay, such as amplification or sequencing to detect for the presence of passenger mutations.

### CROSS REFERENCE TO RELATED APPLICATIONS

This application is a continuation-in-part of U.S. Non-Provisional application Ser. No. 16/018,926, filed Jun. 26, 2018, which claims the benefit of, and priority to, U.S. Provisional Application No. 62/526,091, filed Jun. 28, 2017, and U.S. Provisional Application No. 62/672,217, filed May 16, 2018, the contents of each of which are incorporated by reference.

### TECHNICAL FIELD

The invention relates to molecular genetics.

### BACKGROUND

Cancer is a disease characterized by genomic instability. For example, thousands of mutations may be identified in just a single gene of an individual with a tumor. A lot of effort has been made to identify which mutations drive tumor formation. A relatively small subset of mutations have been identified ‘driver mutations’, responsible for tumor initiation; whereas the majority of mutations are known as ‘passenger mutations’. Passenger mutations are neutral in the conversion of normal cells to tumor cells, but can be of significant diagnostic value.

Driver mutations, whether specific for a single cancer type or associated with many types of cancer, have been relied on as important biomarkers for cancer. Unfortunately, driver mutations occur at low frequencies, making such mutations difficult to detect. Passenger mutations are more common and are unique to each individual and occur at much higher frequencies than driver mutations; making passenger mutations useful biomarkers.

The detection of passenger mutations has been shown to be indicative of cancer recurrence. Furthermore, passenger mutations have also been used to determine therapeutic response and also to predict which treatment may be most effective for a particular individual.

When testing for mutations, such as driver or passenger mutations, related to cancer, physicians often rely on obtaining a liquid or tissue biopsy from the patient. After obtaining the liquid and tissue biopsy, which may be a painful process for the subject, the liquid or tissue biopsy must be analyzed to detect the presence of the driver or passenger mutations. Conventional methods are time consuming and expensive, making it difficult and a financial hardship to implement regular recurrence monitoring and to detect treatment failure before it is too late.

### SUMMARY

The present invention provides methods for diagnosing cancer, monitoring recurrence, and determining therapeutic response via the detection of passenger mutations using a Cas endonuclease. The invention is also useful to identify therapies and to create patient-specific therapeutic and monitoring strategies. According to the invention, passenger mutations associated with a primary tumor or cancer are cataloged. A Cas endonuclease/guide RNA composition is used to identify passenger mutations subsequent to initial treatment in order to monitor therapeutic response and/or to identify recurrent disease. Once a sufficient number of passenger mutations are known, methods of the invention are also useful to aid in the selection of appropriate therapy.

Methods of the invention require no or minimal sample preparation and, thus can be used to identify passenger mutations directly in a biological sample. According to the invention, once passenger mutations are identified, guide RNA molecules are designed to bind upstream and downstream of the mutation. The guide RNA in association with a Cas endonuclease (a Cas/Guide RNA complex) is introduced to a sample and will bind to the previously-identified passenger mutations. Bound complexes are then pulled out of the sample and/or nucleases or other enzymes are used to degrade unprotected DNA in a method called negative enrichment as disclosed in U.S. Pat. No. 10,081,829, incorporated by reference herein. In some embodiments, the passenger mutations are quantified and compared to the levels obtained from the same individual at an earlier time. In some embodiments, the absence of the passenger mutation in the sample may indicate treatment efficacy. In other embodiments, the reduction in the levels of passenger mutations is indicative of treatment efficacy. In yet other embodiments, the absence of the passenger mutation may be indicative of remission or successful treatment. Treatment options are identified by determining levels of passenger mutation and/or the rate at which passenger mutations are changing. As should be apparent from the foregoing, methods of the invention are useful for individualized diagnostic and therapeutic approaches.

Personalized diagnostic monitoring according to the invention is achieved via analysis of a sample obtained from an individual having cancer. A plurality of passenger mutations are identified via sequencing and comparison to a reference sample from the individual. In other embodiments, passenger mutations are obtained from a patient's clinical record. Guide RNA sequences are prepared with reference to the sequencing information obtained from the individual. The guide RNAs, complexed with Cas endonuclease, are introduced into a biological sample from the individual to bind to the passenger mutation. In certain embodiments, passenger mutations known to be common to a certain type of cancer or group of patients are used in the design of the guide RNAs. It is not necessary to prepare guide RNAs against all passenger mutations known or suspected to be present in a sample; however the value of the diagnostic or recurrence monitoring is enhanced as the number of passenger mutations increases.

As used in the invention, Cas/Guide RNA complexes bind to target nucleic acid flanking a passenger mutation. The Cas endonuclease binds to and protects target nucleic acid even if the passenger mutation is only present as a small fraction of the sample. Thus, methods of the invention are useful when analyzing nucleic acid present in low abundance in a sample such as blood or other bodily fluids. Once captured, the target is analyzed to determine if the passenger mutation is present in the sample. In some embodiments of the invention, a report is provided describing the clinical status of the individual. The presence of one or more passenger mutation in the sample may be indicative of residual disease and can be used to enhance therapeutic options. In other embodiments of the invention, a treatment may be identified and included in the report based on the presence of the passenger mutation in the sample.

Target (i.e., passenger) nucleic acid may be enriched relative to other materials in the sample by any suitable enrichment methods, such as by elution of bound Cas proteins. The target nucleic acid may be enriched by elimination of non-target nucleic acid using, for example, nucleases. Enrichment methods may be used alone or in combination with other enrichment methods. As a non-limiting example, exonuclease digestion may be used alone, or may be used before or after elution of bound Cas proteins. The target nucleic acid may be subject to any suitable detection or analysis assay, such as amplification or sequencing.

Methods and related kits described herein are useful to detect the presence of a target nucleic acid, such as a passenger mutation, in a sample. Due to the nature by which Cas complexes bind nucleic acid, methods may be used even where the target is present only in very small quantities, e.g., even as low as 0.01% frequency of mutant fragments among normal fragments in a sample (i.e., where about 50 copies of a circulating tumor DNA fragment harboring a mutation are present among about 500,000 unrelated fragments of similar size). Thus, methods of the invention may have particular applicability in discovering very rare, yet clinically important, information, such as passenger mutations that are specific to and may be used to detect specific mutations among cell-free DNA, such as passenger mutations among circulating tumor DNA.

In a preferred method, CRISPR/Cas systems and associated guide RNAs are introduced to a biological sample. When used according to methods of the invention, Cas endonuclease—whether catalytically active or inactive—will bind to a target via one or more guide RNA and will protect that target (i.e., stay bound), thereby allowing the target to be obtained out of the sample, either via elution of the captured sequence or by elimination of non-target sequence. In certain aspects, the invention provides methods for detecting a target nucleic acid. Methods include obtaining a sample from a subject, introducing Cas proteins and guide RNA into the serum or plasma, and binding the Cas proteins to ends of a target nucleic acid. The Cas protein may be a Cas endonuclease or a catalytically deficient homolog thereof. The target nucleic acid may then be enriched and isolated from the sample.

The nucleic acid may be any naturally-occurring or artificial nucleic acid. The nucleic acid may be DNA, RNA, hybrid DNA/RNA, peptide nucleic acid (PNA), morpholine and locked nucleic acid (LNA), glycol nucleic acid (GNA), threose nucleic acid (TNA), or Xeno nucleic acid. The RNA may be a subpopulation of RNA, such as mRNA, tRNA, rRNA, miRNA, or siRNA. Preferably the nucleic acid is DNA.

The target or feature of interest may be any feature of a nucleic acid. The feature may be a passenger mutation. For example and without limitation, the passenger mutation may be an insertion, deletion, substitution, inversion, amplification, duplication, translocation, or polymorphism.

The target nucleic acid may be from a sub-population of nucleic acid within the nucleic acid sample. For example, the target nucleic acid may contain cell-free DNA, such as cell-free fetal DNA or circulating tumor DNA. In some embodiments, the sample includes plasma from the subject and the target nucleic acid is cell-free DNA (cfDNA). The plasma may be maternal plasma and the target may be of fetal DNA. In certain embodiments, the sample includes plasma from the subject and the target is circulating tumor DNA (ctDNA). In some embodiments, the sample includes at least one circulating tumor cell from a tumor and the target is ctDNA from the tumor cell. In some embodiments, the target nucleic acid is complementary DNA (cDNA), which is made by reverse transcribing RNA. In some embodiments, detecting cDNA is a way to detecting target RNA.

The target nucleic acid may be from any source of nucleic acid. In preferred embodiments, the target is from a biological sample from a human. In preferred embodiments, the bodily fluid sample is a liquid or bodily fluid from a subject, such as bile, blood, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, vomit, or the like. In preferred embodiments, the bodily fluid sample is a blood sample, serum sample, plasma sample, urine sample, saliva sample, semen sample, feces sample, phlegm sample, or liquid biopsy. The sample may be a tissue sample from an animal, such as skin, conjunctiva, gastrointestinal tract, respiratory tract, vagina, placenta, uterus, oral cavity or nasal cavity. The sample may be a liquid biopsy or a tissue biopsy.

In some embodiments, methods include obtaining a biological sample in a collection tube. In a non-limiting example, the bodily fluid is blood and the collection tube is centrifuged to isolate serum or plasma from blood cells. The Cas endonuclease or catalytically deficient homolog thereof is introduced into the serum or plasma. In an embodiment, the Cas endonuclease, or the catalytically deficient homolog thereof, is introduced into the serum or plasma as a ribonucleoprotein (RNP) in which the endonuclease is complexed with the guide RNA. Preferably, the guide RNA includes at least two single guide RNA molecules that each complex with a Cas endonuclease and guide the Cas endonuclease to hybridize to one of the target, wherein the target includes a loci know to harbor a cancer-associated mutation. In preferred embodiments, the cancer-associated mutation is a passenger mutation.

Methods of the invention may include separating the protein-bound target nucleic acid from some or all of the unbound nucleic acid. For example, methods may include binding the protein-bound target nucleic acid to a particle. The particle may include magnetic or paramagnetic material. The method may include applying a magnetic field to the sample. The particle may include an agent that binds to a protein bound to an end of the target nucleic acid.

The agent may an antibody or fragment thereof. The method may include chromatography, applying the sample to a column, or gel electrophoresis. The method may include separating the protein-bound target nucleic acid from some or all of the unbound nucleic acid by size exclusion, ion exchange, or adsorption.

Each of the proteins may independently be any protein that binds a nucleic acid in a sequence-specific manner. The protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). The protein may be a catalytically inactive form of a nuclease, such as a programmable nuclease described above. The protein may be a transcription activator-like effector (TALE). The protein may be complexed with a nucleic acid that guides the protein to an end of the nucleic acid. For example, the protein may be a Cas endonuclease in a complex with one or more guide RNAs. Preferably, the protein is a Cas endonuclease or a catalytically deficient homolog thereof.

The target nucleic acid may be detected by any means known in the art. For example and without limitation, the target nucleic acid may be detected by DNA staining, spectrophotometry, sequencing, fluorescent probe hybridization, fluorescence resonance energy transfer, optical microscopy, or electron microscopy. Detecting the target nucleic acid may include identifying a passenger mutation in the target nucleic acid. Identifying the passenger mutation may include sequencing the nucleic acid (e.g., on a next-generation sequencing instrument), allele-specific amplification, and hybridizing a probe to the nucleic acid.

Methods of the invention may include amplifying the target nucleic acid to yield amplicons. Methods may further include sequencing the target nucleic acid to produce sequence reads and analyzing the sequence reads to provide genetic information of the subject. Methods may include analyzing the target nucleic acid to describe one or more passenger mutations in the subject. Methods may further include analyzing the nucleic acid to monitor cancer reoccurrence in the individual. In other embodiments, methods may include analyzing the nucleic acid to assess treatment efficacy. In yet other embodiments, analyzing the nucleic acid may include identifying a treatment.

In some embodiments, the target nucleic acid includes a passenger mutation specific to a tumor. The target nucleic acid may be present at no more than about 0.01% of cell-free DNA in the plasma or serum. By methods herein, the target nucleic acid is isolated or enriched from the serum or plasma.

Certain methods may further include detecting the target nucleic acid (e.g., by amplification, sequencing, probe hybridization, digital PCR, etc.). For example, detecting the target nucleic acid may include hybridizing the target nucleic acid to a probe or to a primer for a detection or amplification step, or labelling the target nucleic acid with a detectable label. Because the Cas proteins may be used to bind to the target in a sequence-specific manner, and thereby isolate or enrich for a specific passenger mutation, detecting the presence of the nucleic acid may be useful to report the presence of the passenger mutation in a subject from whom the sample is obtained. In multiplexed embodiments, a panel, or any number of passenger mutations, is assayed for through use of steps of the methods and the results may provide a count or description of passenger mutations detected from the target nucleic acid in the sample.

Furthermore, methods of the invention may include negative enrichment. As an example, Cas endonuclease may be provided with one or more guide RNAs that bind to a target nucleic acid and flank a loci of interest, such as a locus of the known cancer-associated passenger mutation. The Cas endonuclease bind to, and protect, passenger mutation-containing nucleic acid even when the passenger mutation is only present as a small fraction of the sample. The bound Cas proteins prevent exonuclease from digesting the target nucleic acid and, after incubation with exonuclease, the only nucleic acid substantially present in the sample is the target nucleic acid. The target nucleic acid is thus isolated or enriched in a sequence-specific manner. The target nucleic acid may then be subject to any suitable detection or analysis assay such as amplification or sequencing.

In a preferred method, CRISPR/Cas systems using guide RNAs specific for a passenger mutation is introduced to the sample under conditions such that nucleic acid containing the passenger mutation is protected from exonuclease digestion while non-target nucleic acid is digested by an exonuclease. When used according to methods of the invention, Cas endonuclease—whether catalytically active or inactive—will bind to a target consistently via a guide RNA and will protect that target (i.e., stay bound) for at least long enough that a promiscuous exonuclease can be reliably used to digest unbound, non-target nucleic acid. By protection of the target with digestion of the non-target, a sample is effectively enriched for the target, and those remaining target fragments are captured, stored, isolated, preserved, detected, sequenced, or otherwise assayed with success that would be unobtainable without methods of the invention.

### BRIEF DESCRIPTION OF THE DRAWINGS

FIG. 1 shows a table of the inputs and the dilation amounts used in the Example described herein. Dilution 11 is at 3× concentration from previous experiments because the experiment uses 3× as much input DNA volume in the reaction. The copies per ul of stock, copies per ul in 50 ul reaction, amount of previous dilution (ul), plasma, and total volume (ul) are indicated.

FIG. 2 shows a table of the dilutions used in the Example. For the percent of plasma in the final reaction, the percent of plasma in 2× sample, plasma dilution (ul), and tris dilution (ul) are shown in the table.

FIG. 3 shows a graph of the qPCR results after amplification from the post-cutting dilutions described in the Example.

FIG. 4 shows the tabulated qPCR results from the Example. Percent plasma, use of a Streck tube, amount of no Cas9 present, amount of Cas9 present, and percent cutting are indicated.

FIG. 5 shows a chart of the binding efficiency from the Example, particularly showing the relationship between percent cleavage and percent plasma. In particular, the percent cleavage is shown as a function of the amount or percent of plasma in the cutting reaction. Results are shown for samples with no tube and samples using a Streck tube.

FIG. 6 shows a chart of the detection signal in plasma from the Example, particularly showing the relationship between qPCR signal and percent plasma. In particular, the percent detection of no plasma in the sample is shown as a function of the percent plasma in the cutting reaction. Results are shown for samples with no tube and sample using a Streck tube.

### DETAILED DESCRIPTION

Methods of the invention are useful for the detection of passenger mutations directly from biological samples without the need for complex sample preparation. The sample is enriched for the target nucleic acid and passenger mutations are detected. In preferred embodiments of the invention, the detection of passenger mutations is indicative of cancer recurrence. In other embodiments, treatment efficacy is assessed by detecting for the presence or absence of passenger mutations. In yet other embodiments, treatments are identified by detecting and analyzing the mutations.

Methods of the invention include introducing the Cas endonuclease, catalytically inactive Cas endonuclease, or homolog thereof and guide RNA into the bodily fluid sample. In a preferred embodiment, the binding proteins are provided by Cas endonuclease/guide RNA complexes. Embodiments of the invention use Cas endonuclease proteins that are originally encoded by genes that are associated with clustered regularly interspaced short palindromic repeats (CRISPR) in bacterial genomes. A CRISPR-associated (Cas) endonuclease may be introduced directly into the bodily fluid sample.

Methods of the invention include utilizing the complexes for monitoring cancer recurrence and/or therapeutic efficacy. In embodiments of the invention, passenger mutations are identified in a biological sample. The passenger mutations may be specific to a tumor of an individual. The detection of the passenger mutations in a sample is not only indicative of the presence or recurrence of cancer in the individual, but is also useful to determine therapeutic efficacy. Methods of the invention provide for detecting the presence of a tumor by detecting passenger mutations directly in a body fluid sample without the need for significant sample preparation steps or kits. Methods of the invention use Cas endonuclease to bind target nucleic acid sequences of passenger mutations of interest.

Methods of the invention identify passenger mutations specific to an individual tumor. In some embodiments of the invention, a passenger mutation may be identified by obtaining a sample from an individual with cancer. Sequencing is performed on the sample to determine one or more passenger mutations in the reference sample. Ideally, this is done with respect to a reference (i.e., non-tumor) sample obtained from the same patient. In other embodiments, the passenger mutation is obtained from clinical data of the individual. The clinical data may be obtained from a plurality of sources, such as laboratory test reports. In yet other embodiments, sequence data of a driver mutation may also be obtained from clinical data of the individual. In some embodiments, sequence data of the passenger mutation is stored in a database and catalogued. A guide RNA sequence may be identified using the sequence data of the passenger mutation. The guide RNA complexed with Cas endonuclease may then be introduced into a biological sample from the individual to bind to the passenger mutation. Methods of the invention include identifying passenger mutations indicative of the presence of cancer in the individual.

Methods of the invention provide for monitoring cancer by obtaining a sample at one or more time points after treatment. For example, the sample may be obtained during treatment, after treatment, or during remission. The sample is a biological sample, such as a bodily fluid sample. In certain embodiments, the sample may be obtained from the individual subsequent to the individual starting a treatment.

Using sequence specific DNA-binding proteins, such as Cas endonuclease, to bind target nucleic acid sequences of interest, such as those that contain the passenger mutation of the individual, cancer recurrence can be monitored. The Cas endonuclease is provided with one or more guide RNAs that bind to the target nucleic acid that includes or flank a locus of the passenger mutation of the individual. The Cas endonuclease binds to and protects target nucleic acid even when the passenger mutation is only present as a small fraction of the sample. Thus, methods of the invention are useful when analyzing nucleic acid present in low abundance in a sample such as blood or other bodily fluids.

In methods of the invention, the Cas proteins bind to ends of a target nucleic acid. The target nucleic acid is thus isolated or enriched in a sequence-specific manner. The enriched target nucleic acid may then be subject to any suitable detection or analysis assay such as amplification or sequencing. The enriched target nucleic acid may be further enriched by digesting other, unbound nucleic acids present in the sample with exonuclease. The bound Cas proteins prevent the exonuclease from digesting the target nucleic acid, thereby leaving the only the target nucleic acid substantially present in the sample. The target nucleic acid is thus isolated or enriched in a sequence-specific manner. The target nucleic acid may then be subject to any suitable detection or analysis assay such as amplification or sequencing.

Preferably, the Cas endonuclease is complexed with a guide RNA that targets the Cas endonuclease to a specific sequence. Any suitable Cas endonuclease or homolog thereof may be used. A Cas endonuclease (catalytically active or deactivated) may be Cas9 (e.g., spCas9), catalytically inactive Cas (dCas such as dCas9), Cpf1 (aka Cas12a), C2c2, Cas13, Cas13a, Cas13b, e.g., PsmCas13b, LbaCas13a, LwaCas13a, AsCas12a, others, modified variants thereof, and similar proteins or macromolecular complexes. The Cas13 proteins may be preferred where the target includes RNA. A Cas endonuclease/guide RNA complex includes a first Cas endonuclease and a first guide RNA. In the depicted embodiment, the complex comprises the Cas endonuclease or the catalytically deficient homolog thereof being introduced into the serum or plasma as a ribonucleoprotein (RNP) in which the Cas endonuclease or catalytically deficient homolog thereof is complexed with the guide RNA. The Cas endonuclease will bind to the target. The target may then be isolated or enriched, allowing for detection of the target.

The proteins that bind to ends of the target nucleic acid may be any proteins that bind to a nucleic acid in a sequence-specific manner. The protein may be a programmable nuclease. For example, the protein may be a CRISPR-associated (Cas) endonuclease, zinc-finger nuclease (ZFN), transcription activator-like effector nuclease (TALEN), or RNA-guided engineered nuclease (RGEN). Programmable nucleases and their uses are described in, for example, Zhang, 2014, “CRISPR/Cas9 for genome editing: progress, implications and challenges”, Hum Mol Genet 23 (R1):R40-6; Ledford, 2016. CRISPR: gene editing is just the beginning, Nature. 531 (7593): 156-9; Hsu, 2014, Development and applications of CRISPR-Cas9 for genome engineering, Cell 157(6):1262-78; Boch, 2011, TALEs of genome targeting, Nat Biotech 29(2):135-6; Wood, 2011, Targeted genome editing across species using ZFNs and TALENs, Science 333(6040):307; Carroll, 2011, Genome engineering with zinc-finger nucleases, Genetics Soc Amer 188(4):773-782; and Urnov, 2010, Genome Editing with Engineered Zinc Finger Nucleases, Nat Rev Genet 11(9):636-646, each incorporated by reference.

The protein may be a catalytically inactive form of a nuclease, such as a programmable nuclease described above. The protein may be a transcription activator-like effector (TALE). The protein may be complexed with a nucleic acid that guides the protein to an end of the target nucleic acid. For example, the protein may be a Cas endonuclease in a complex with one or more guide RNAs. In preferred embodiments, the protein is a Cas endonuclease, catalytically inactive Cas endonuclease, or homologs thereof.

In certain embodiments, the sample includes cfDNA from a subject. The sample is exposed to a first Cas endonuclease/guide RNA complex that binds to a target nucleic acid (e.g., a passenger mutation of interest) in a sequence-specific fashion. In some embodiments, the complex binds to a mutation in a sequence-specific manner. A segment of the nucleic acid, i.e., the target nucleic acid, is protected by introducing the first Cas endonuclease/guide RNA complex and a second Cas endonuclease/guide RNA complex that also binds to the nucleic acid. In preferred embodiments of the method, the guide RNA comprises at least two guide RNA molecules that each complex with a Cas endonuclease and guide the Cas endonuclease to hybridize to one target nucleic acid, wherein the target nucleic acid includes a loci know to harbor a cancer-associated mutation. In other preferred embodiments of the method, the guide RNA comprises at least two guide RNA molecules that each complex with a Cas endonuclease and guide the Cas endonuclease to hybridize to one target nucleic acid, wherein the target nucleic acid includes a loci know to harbor a cancer-associated passenger mutation specific to the individual.

Optionally, unprotected nucleic acid is digested. For example, one or more exonucleases may be introduced that promiscuously digest unbound, unprotected nucleic acid. Any suitable exonuclease may be used. Suitable exonucleases include, for example, Lambda exonuclease, RecJf, Exonuclease III, Exonuclease I, Exonuclease T, Exonuclease V, Exonuclease VII, T5 Exonuclease, and T7 Exonuclease, most of which are available from New England Biolabs (Ipswich, Mass.). While the exonucleases act, the target nucleic acid is protected by the bound complexes and survives the digestion step intact.

The described steps including the digestion by the exonuclease leave a reaction product that includes principally only the mutant segment of nucleic acid, as well as any spent reagents, Cas endonuclease complexes, exonuclease, nucleotide monophosphates, and pyrophosphate as may be present.

In certain embodiments, the exonuclease is deactivated. For example, exonuclease may be deactivated by following the manufacturer's instructions e.g., by heating to 90 degrees for a few minutes. After digestion, a positive selection step may be performed which may include, for example, amplification of the target nucleic acid by known methods or selection by an affinity assays.

The nucleic acid may be any naturally-occurring or artificial nucleic acid. The nucleic acid may be DNA, RNA, hybrid DNA/RNA, peptide nucleic acid (PNA), morpholine and locked nucleic acid (LNA), glycol nucleic acid (GNA), threose nucleic acid (TNA), or Xeno nucleic acid. The RNA may be a subpopulation of RNA, such as mRNA, tRNA, rRNA, miRNA, or siRNA. Preferably the nucleic acid is DNA.

The target or feature of interest may be any feature of a nucleic acid. The feature may be a mutation. For example and without limitation, the feature may be an insertion, deletion, substitution, inversion, amplification, duplication, translocation, or polymorphism. The feature may be a nucleic acid from an infectious agent or pathogen. For example, the nucleic acid sample may be obtained from an organism, and the feature may contain a sequence foreign to the genome of that organism. In a preferred embodiment, the feature of interest is a passenger mutation. In other embodiments, the feature of interest is a passenger mutation signature.

The target nucleic acid may be from a sub-population of nucleic acid within the nucleic acid sample. For example, the target nucleic acid may contain cell-free DNA, such as cell-free fetal DNA or circulating tumor DNA. In some embodiments, the sample includes plasma from the subject and the target nucleic acid is cell-free DNA (cfDNA). The plasma may be maternal plasma and the target may be of fetal DNA. In certain embodiments, the sample includes plasma from the subject and the target is circulating tumor DNA (ctDNA). In some embodiments, the sample includes at least one circulating tumor cell from a tumor and the target is tumor DNA from the tumor cell. In some embodiments, the target nucleic acid is complementary DNA (cDNA), which is made by reverse transcribing RNA. In some embodiments, detecting cDNA is a way to detecting target RNA.

The target nucleic acid may be from any source of nucleic acid. In preferred embodiments, the target nucleic acid is from a bodily fluid sample from a human. In preferred embodiments, the bodily fluid sample is a liquid or bodily fluid from a subject, such as bile, blood, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, vomit, or the like. In preferred embodiments, the bodily fluid sample is a blood sample, serum sample, plasma sample, urine sample, saliva sample, semen sample, feces sample, phlegm sample, or liquid biopsy. The sample may be a tissue sample from an animal, such as skin, conjunctiva, gastrointestinal tract, respiratory tract, vagina, placenta, uterus, oral cavity or nasal cavity. The sample may be a liquid biopsy or a tissue biopsy.

The method optionally includes detecting the target nucleic acid (which may harbor the mutation). Any suitable technique may be used to detect the target nucleic acid. For example, detection may be performed using DNA staining, spectrophotometry, sequencing, fluorescent probe hybridization, fluorescence resonance energy transfer, optical microscopy, electron microscopy, others, or combinations thereof. Detecting the target nucleic acid may indicate the presence of the mutation in the subject (i.e., a patient), and a report may be provided describing the mutation in the patient. In a preferred embodiment, detecting the target nucleic acid in the sample may indicate the presence of the passenger mutation in the subject, and a report may be provided describing the passenger mutation in the subject. The report may describe the recurrence of cancer in the subject. In other embodiments, the report may identify a treatment based on the presence of the passenger mutation in the sample. In other embodiments, the report may describe the efficacy of a treatment. In yet other embodiments, the report may describe resistance to a treatment based on the presence of the passenger mutation in the sample.

In an embodiment of the invention, a sample may contain a mutant fragment of DNA, a wild-type fragment of DNA, or both. A locus of interest is identified where a mutation may be present proximal to, or within, a protospacer adjacent motif (PAM). When the wild-type fragment is present, it may contain a wild-type allele at a homologous location in the fragment, also proximal to, or within, a PAM. A guide RNA is introduced to the sample that has a targeting portion complementary to the portion of the mutant fragment that includes the mutation. When a Cas endonuclease is introduced, it will form a complex with the guide RNA and bind to the mutant fragment but not to the wild-type fragment. The first Cas endonuclease/guide RNA complex includes a guide RNA with a targeting region that binds to the mutation but that does not bind to other variants at a loci of the mutation. The described methodology may be used to target a mutation that is proximal to a PAM, or it may be used to target and detect a mutation in a PAM, e.g., a loss-of-PAM or gain-of-PAM mutation.

The described methodology may be used to target a mutation that is proximal to a PAM, or it may be used to target and detect a mutation in a PAM, e.g., a loss-of-PAM or gain-of-PAM mutation. The PAM is typically specific to, or defined by, the Cas endonuclease being used. For example, for Streptococcus pyogenes Cas9, the PAM includes NGG, and the targeted portion includes the 20 bases immediately 5′ to the PAM. As such, the targetable portion of the DNA includes any twenty-three consecutive bases that terminate in GG or that are mutated to terminate in GG. Such a pattern may be found to be distributed over ctDNA at such frequency that the potentially detectable mutations are abundant enough as to be representative of mutations over the tumor DNA at large. In such cases, mutation-specific enrichment may be used to detect mutations from a tumor. Moreover, methods may be used to determine a number of mutations over the representative, targetable portion of tumor DNA. Since the targetable portion of the genome is representative of the tumor DNA overall, the number of mutations may be used to infer a mutational burden for the tumor.

A feature of the method is that a specific mutation may be detected by a technique that includes detecting only the presence or absence of a fragment of DNA, and it need not be necessary to sequence DNA from a subject to describe mutations. Preferably, the passenger mutation is detected in a sample by such methods of the invention. Methods of the invention use protection at one or both ends of DNA segments. The gRNA selects for a known mutation on one end. A positive selection may be performed to positively select out the bound, target nucleic acid. If the gRNA does not find the mutation, no protection is provided and the molecule may be digested, e.g. in negative enrichment, and the remaining molecules are either counted or sequenced. Methods are well suited for the analysis of samples in which the target of interest is extremely rare, and particularly for the analysis of maternal plasma or serum (e.g., for fetal DNA) or a liquid biopsy (e.g., for ctDNA).

Methods are useful for the isolation of intact DNA fragments of any arbitrary length and may preferably be used in some embodiments to isolate (or enrich for) arbitrarily long fragments of DNA, e.g., tens, hundreds, thousands, or tens of thousands of bases in length or longer. Long, isolated, intact fragments of DNA may be analyzed by any suitable method such as simple detection (e.g., via staining with ethidium bromide) or by single-molecule sequencing. It is noted that the Cas9/gRNA complexes may be subsequently or previously labeled using standard procedures. The complexes may be fluorescently labeled, e.g., with distinct fluorescent labels such that detecting involves detecting both labels together (e.g., after a dilution into fluid partitions). Preferred embodiments of the detection do not require PCR amplification and therefore significantly reduces cost and sequence bias associated with PCR amplification. Sample analysis can also be performed by a number of approaches, such as next generation sequencing (NGS), etc. However, many analytical platforms may require PCR amplification prior to analysis. Therefore, preferred embodiments of analysis of the reaction products include single molecule analysis that avoids the requirement of amplification.

Kits and methods of the invention are useful with methods disclosed in U.S. Provisional Patent Application 62/526,091, filed Jun. 28, 2017, for POLYNUCLEIC ACID MOLECULE ENRICHMENT METHODOLOGIES and U.S. Provisional Patent Application 62/519,051, filed Jun. 13, 2017, for POLYNUCLEIC ACID MOLECULE ENRICHMENT METHODOLOGIES, both incorporated by reference.

The target nucleic acid may be detected, sequenced, or counted. Where a plurality of fragments are present or expected, the fragment may be quantified, e.g., by qPCR. Passenger mutations may be quantified by methods known in the art. Expression levels of the passenger mutation in the sample may be compared to expression levels of the passenger mutation in subsequent samples. In a preferred embodiment, the efficacy of a treatment is determined. The reduction of the expression level of the passenger mutation in the sample is indicative of treatment efficacy.

The target nucleic acid may further be isolated or detected by any suitable method in order to separate the target segment from other nucleic acids in the sample. For example, the isolation or detection method may include separating the protein-bound target nucleic acid from some or all of the unbound nucleic acid. The isolation or detection method may include binding the protein-bound target nucleic acid to a particle. The particle may include magnetic or paramagnetic material. The isolation or detection method may include applying a magnetic field to the sample. The particle may include an agent that binds to a protein bound to an end of the target nucleic acid. The agent may an antibody or fragment thereof. The isolation or detection method may include chromatography. The isolation or detection method may include applying the sample to a column. The isolation or detection method may include separating the protein-bound target nucleic acid from some or all of the unbound nucleic acid by size exclusion, ion exchange, or adsorption. The isolation or detection method may include gel electrophoresis.

Embodiments of the invention may include detecting the target nucleic acid and optionally providing a report describing a mutation as present in the patient. The mutation-containing fragments may be detected by a suitable assay, such as sequencing, gel electrophoresis, a probe-based assay. The detection of the isolated segment of the target nucleic acid may be done by sequencing. The digestion provides a reaction product that includes principally only the target nucleic acid, as well as any spent reagents, Cas endonuclease complexes, exonuclease (e.g. when negative enrichment is performed), nucleotide monophosphates, or pyrophosphate as may be present. The reaction product may be provided as an aliquot (e.g., in a micro centrifuge tube such as that sold under the trademark EPPENDORF by Eppendorf North America (Hauppauge, N.Y.) or glass cuvette). The reaction product aliquot may be disposed on a substrate. For example, the reaction product may be pipetted onto a glass slide and subsequently combed or dried to extend the fragment across the glass slide. The reaction product may optionally be amplified. Optionally, adaptors are ligated to ends of the reaction product, which adaptors may contain primer sites or sequencing adaptors. The presence of the segment in the reaction product aliquot may then be detected using an instrument.

The target nucleic acid may be detected by any means known in the art. For example and without limitation, the target nucleic acid may be detected by DNA staining, spectrophotometry, sequencing, fluorescent probe hybridization, fluorescence resonance energy transfer, optical microscopy, or electron microscopy. Detecting the nucleic acid may include identifying a mutation in the nucleic acid. The mutation may be a passenger mutation specific to an individual with cancer. Identifying the mutation may include sequencing the nucleic acid (e.g., on a next-generation sequencing instrument), allele-specific amplification, and hybridizing a probe to the nucleic acid. Methods of DNA sequencing are known in the art and described in, for example, Peterson, 2009, Generations of sequencing technologies, Genomics 93(2):105-11; Goodwin, 2016, Coming of age: ten years of next-generation sequencing technologies, Nat Rev Genet 17(6):333-51; and Morey, 2013, A glimpse into past, present, and future DNA sequencing, Mol Genet Metab 110(1-2):3-24, each incorporated by reference. Other methods of DNA detection are known in the art and described in, for example, Xu, 2014, Label-Free DNA Sequence Detection through FRET from a Fluorescent Polymer with Pyrene Excimer to SG, ACS Macro Lett 3(9):845-848, incorporated by reference.

One method for detection of protein-bound nucleic acids is immunomagnetic separation. Magnetic or paramagnetic particles are coated with an antibody that binds the protein bound to the segment, and a magnetic field is applied to separate particle-bound segment from other nucleic acids. Methods of immunomagnetic purification of biological materials such as cells and macromolecules are known in the art and described in, for example, U.S. Pat. No. 8,318,445; Safarik and Safarikova, Magnetic techniques for the isolation and purification of proteins and peptides, Biomagn Res Technol. 2004; 2:7, doi: 10.1186/1477-044X-2-7, the contents of each of which are incorporated herein by reference. The antibody may be a full-length antibody, a fragment of an antibody, a naturally occurring antibody, a synthetic antibody, an engineered antibody, or a fragment of the aforementioned antibodies. Alternatively or additionally, the particles may be coated with another protein-binding moiety, such as an aptamer, peptide, receptor, ligand, or the like.

Chromatographic methods may be used for detection. In such methods, the bodily fluid sample is applied to a column, and the target nucleic acid is separated from other nucleic acids based on a difference in the properties of the target nucleic acid and the other nucleic acids. Size exclusion chromatography is useful for separating molecules based on differences in size and thus is useful when the segment is larger than other nucleic acids, for example the residual nucleic acids left from a digestion step. Methods of size exclusion chromatography are known in the art and described in, for example, Ballou, David P.; Benore, Marilee; Ninfa, Alexander J. (2008). Fundamental laboratory approaches for biochemistry and biotechnology (2nd ed.). Hoboken, N.J.: Wiley. p. 129. ISBN 9780470087664; Striegel, A. M.; and Kirkland, J. J.; Yau, W. W.; Bly, D. D.; Modern Size Exclusion Chromatography, Practice of Gel Permeation and Gel Filtration Chromatography, 2nd ed.; Wiley: NY, 2009, the contents of each of which are incorporated herein by reference.

Ion exchange chromatography uses an ion exchange mechanism to separate analytes based on their respective charges. Thus, ion exchange chromatography can be used with the proteins bound to the target nucleic acid impart a differential charge as compared to other nucleic acids. Methods of ion exchange chromatography are known in the art and described in, for example, Small, Hamish (1989). Ion chromatography. New York: Plenum Press. ISBN 0-306-43290-0; Tatjana Weiss, and Joachim Weiss (2005). Handbook of Ion Chromatography. Weinheim: Wiley-VCH. ISBN 3-527-28701-9; Gjerde, Douglas T.; Fritz, James S. (2000). Ion Chromatography. Weinheim: Wiley-VCH. ISBN 3-527-29914-9; and Jackson, Peter; Haddad, Paul R. (1990). Ion chromatography: principles and applications. Amsterdam: Elsevier. ISBN 0-444-88232-4, the contents of each of which are incorporated herein by reference.

Adsorption chromatography relies on difference in the ability of molecule to adsorb to a solid phase material. Larger nucleic acid molecules are more adsorbent on stationary phase surfaces than smaller nucleic acid molecules, so adsorption chromatography is useful when the target nucleic acid is larger than other nucleic acids, for example the residual nucleic acids left from a digestion step. Methods of adsorption chromatography are known in the art and described in, for example, Cady, 2003, Nucleic acid purification using microfabricated silicon structures. Biosensors and Bioelectronics, 19:59-66; Melzak, 1996, Driving Forces for DNA Adsorption to Silica in Perchlorate Solutions, J Colloid Interface Sci 181:635-644; Tian, 2000, Evaluation of Silica Resins for Direct and Efficient Extraction of DNA from Complex Biological Matrices in a Miniaturized Format, Anal Biochem 283:175-191; and Wolfe, 2002, Toward a microchip-based solid-phase extraction method for isolation of nucleic acids, Electrophoresis 23:727-733, each incorporated by reference.

Another method for detection is gel electrophoresis. Gel electrophoresis allows separation of molecules based on differences in their sizes and is thus useful when the target nucleic acid is larger than other nucleic acids, for example the residual nucleic acids left from a digestion step. Methods of gel electrophoresis are known in the art and described in, for example, Tom Maniatis; E. F. Fritsch; Joseph Sambrook. “Chapter 5, protocol 1”. Molecular Cloning - A Laboratory Manual. 1 (3rd ed.). p. 5.2-5.3. ISBN 978-0879691363; and Ninfa, Alexander J.; Ballou, David P.; Benore, Marilee (2009). fundamental laboratory approaches for biochemistry and biotechnology. Hoboken, NJ: Wiley. p. 161. ISBN 0470087668, the contents of which are incorporated herein by reference.

Certain preferred embodiments include obtaining a blood, plasma, or serum sample from a patient. In certain embodiments, a sample is obtained from an individual subsequent to treatment of the cancer. The blood, plasma, or serum may include cfDNA and thus also include ctDNA among the cfDNA. Specific sequences of the ctDNA are isolated or enriched and analyzed or detected to detect or report genetic information from the subject, such as a presence or count of certain tumor mutations. Methods of the invention include introduce Cas endonucleases (or catalytically inactive homologs thereof such as dCas9) directly into serum or plasma. The Cas endonucleases are complexed with guide RNAs that include targeting portions specific for a target nucleic acid. In the plasma or serum, the complexes bind to ends of the target and protect it. Exonuclease may be introduced to digest unbound nucleic acid into monomers and fragments too small for further meaningful detection, sequencing, or amplification. In preferred embodiments, the Cas/guide RNA complex are introduced directly into the subsequent sample.

Embodiments of the invention provide for treatment of a sample. For example, a blood sample may be obtained from a patient. The sample may be collected in any suitable blood collection tube such as the collection tube sold under the trademark VACUTAINER by BD (Franklin Lakes, N.J.). In certain embodiments, the collection tube comprises an EDTA collection tube, and Na-EDTA collection tube or the collection tube sold under the trademark CELL-FREE DNA BCT by Streck, Inc. (La Vista, Nebr.), sometimes referred to in the art as a Streck tube. Use of a Streck tube stabilizes nucleated blood cells and prevents the release of genomic DNA into the sample. This facilitates the collection of sample that includes cell-free DNA.

The sample may be centrifuged to generate a sample that includes a pellet of blood cells and a supernatant, which contains serum or plasma. Serum is the liquid supernatant of whole blood that is collected after the blood is allowed to clot and centrifuged. Plasma is produced when the process includes an anticoagulant. To collect serum, blood is collected in tubes. After collection, the blood is allowed to clot by leaving it undisturbed at room temperature (about 15-30 minutes). The clot is removed by centrifuging, e.g., at 1,000-2,000×g for 10 minutes in a refrigerated centrifuge. The resulting supernatant is designated serum and may be transferred to a clean polypropylene tube using a Pasteur pipette. For plasma, blood is collected into commercially available anticoagulant-treated tubes e.g., EDTA-treated (lavender tops), citrate-treated (light blue tops), or heparinized tubes (green tops), followed by centrifugation to collect the supernatant. The supernatant is preferably transferred to a fresh tube, away from the pellet, which may be discarded. Particularly where the collection tube included an anticoagulant, the transfer should give a good separation of the plasma from the whole blood cells. After transfer, the sample includes plasma or serum, which includes cfDNA.

In an exemplary embodiment, serum or plasma is transferred from a centrifuge tube to a new tube, complexes comprising Cas9 and guide RNA are added, and the mixture is incubated. For example, amplification or an affinity assay may be performed to positively select out the bound, target nucleic acid. In another embodiment, exonuclease may be introduced to digest unbound, non-target DNA, and then the exonuclease may be deactivated (e.g., by heat). A positive selection may then follow (e.g., amplification or an affinity assay) to positively select out the bound, target nucleic acid.

In another exemplary embodiment, plasma or serum is removed from the centrifuge tube (the supernatant) and transferred into a new tube. Appropriate buffers/reagents are added to modify a chemical environment to promote binding of Cas endonuclease to the target nucleic acid. For example, pH can be adjusted, as may temperature, salinity, or co-factors present. The Cas complexes are added and allowed to incubate. For example, amplification or an affinity may be performed to positively select out the bound, target nucleic acid. An exonuclease may optionally be added, which ablates all free, non-target nucleic acid. The target may be positively selected such as by amplification or an affinity assay after exonuclease digestion of the non-target nucleic acid.

Methods may include detection or isolation of circulating tumor cells (CTCs) from a blood sample. Cytometric approaches use immunostaining profiles to identify CTCs. CTC methods may employ an enrichment step to optimize the probability of rare cell detection, achievable through immune-magnetic separation, centrifugation, or filtration. Cytometric CTC technology includes the CTC analysis platform sold under the trademark CELLSEARCH by Veridex LLC (Huntingdon Valley, Pa.). Such systems provide semi-automation and proven reproducibility, reliability, sensitivity, linearity and accuracy. See Krebs, 2010, Circulating tumor cells, Ther Adv Med Oncol 2(6):351-365 and Miller, 2010, Significance of circulating tumor cells detected by the CellSearch system in patients with metastatic breast colorectal and prostate cancer, J Oncol 2010:617421-617421, both incorporated by reference.

Certain embodiments of the invention may provide a kit. The kit preferably includes reagents and materials useful for performing methods of the invention. For example, the kit may include one or more guide RNA that, taken in pairs, are designed to flank cancer-associated mutations. The kit may include one or more guide RNAs that are mutation specific and only hybridize to target that includes a mutation. The kit may include a Cas endonuclease or a nucleic acid encoding a Cas endonuclease such as a plasmid. The kit may optionally include exonuclease. The kit may include reagents for adjusting conditions such as pH, salinity, co-factors, etc., to promote binding or activity of Cas endonuclease (including to promote binding of catalytically inactive Cas endonuclease, which may be included as the Cas endonuclease) in the bodily fluid sample, such as plasma or serum. The kit may further include instructional materials for performing methods of the invention, and components of the kit may be packaged in a box suitable for shipping or storage. Preferably, the kit contains one or more collection tubes, such as a blood collection tube. In other embodiments, the kit may include one or more guide RNA that, taken in pairs, are designed to flank cancer-associated passenger mutation sites specific to an individual.

The Cas endonuclease/guide RNA complexes can be designed to bind to mutations of clinical significance, such as a mutation specific to a tumor. In other embodiments, the complexes can be designed to bind to passenger mutations of individual clinical significance. When a mutation is thus detected, a report may be provided to, for example, describe the mutation in a patient or a subject. Thus, certain embodiments may comprise providing a report. The report preferably includes a description of the mutation in the subject (e.g., a patient). The method for detecting rare nucleic acid may be used in conjunction with a method of describing mutations (e.g., as described herein). Either or both detection processes may be performed over any number of loci in a patient's genome or preferably in a patient's tumor DNA. As such, the report may include a description of a plurality of structural alterations, mutations, or both in the patient's genome or tumor DNA. As such, the report may give a description of a mutational landscape of a tumor. In other embodiments, the report may provide a description of cancer reoccurrence. In yet other embodiments, the report may provide a description of the efficacy of a treatment.

Knowledge of a mutational landscape of a tumor may be used to inform treatment decisions, monitor therapy, detect remissions, or combinations thereof. For example, where the report includes a description of a plurality of mutations, the report may also include an estimate of a tumor mutation burden (TMB) for a tumor. It may be found that TMB is predictive of success of immunotherapy in treating a tumor, and thus methods described herein may be used for treating a tumor. In another example, where the report includes a description of the relative expression of the passenger mutations, the report may also include an estimate of treatment efficacy. In yet another example, the report may also include the presence of passenger mutations in a sample, the report may also indicate the recurrence of cancer in the individual. It may be found that passenger mutations and their expression levels may be predictive of specific treatment efficacy and thus methods described herein may be used for identifying a treatment and treating a tumor.

Methods of the invention thus may be used to detect and report clinically actionable information about a patient or a tumor in a patient. For example, the method may be used to provide a report describing the presence of the genomic alteration in a genome of a subject. Additionally, protecting a segment of DNA, and optionally digesting unprotected DNA, provides a method for isolation or enrichment of DNA fragments, i.e., the protected segment. It may be found that the described enrichment techniques are well-suited to the isolation/enrichment of arbitrarily long DNA fragments, e.g., thousands to tens of thousands of bases in length or longer.

Long DNA fragment targeted enrichment, or negative enrichment, creates the opportunity of applying long read platforms in clinical diagnostics. Negative enrichment may be used to enrich “representative” genomic regions that can allow an investigator to identify “off rate” when performing CRISPR Cas9 experimentation, as well as enrich for genomic regions that would be used to determine TMB for immuno-oncology associated therapeutic treatments. In such applications, the negative enrichment technology is utilized to enrich large regions (>50 kb) within the genome of interest.

By the described methods, a bodily fluid sample can be assayed for a mutation using a technique that is inexpensive, quick, and reliable. Methods of the invention are conducive to high throughput embodiments, and may be performed, for example, in droplets on a microfluidic device, to rapidly assay a large number of aliquots from a sample for one or any number of genomic structural alterations. Furthermore, using the methods described herein, the monitoring of cancer recurrence specific to an individual is obtained by detecting for passenger mutations specific to an individual in a sample obtained from an individual from time to time.

### EXAMPLE

The cutting efficiency of amplicons by Cas9 in plasma is shown by experiment. Results from the experiment indicated that Cas proteins bind to expected cognate targets under guide RNA guidance in plasma or serum. In particular, Cas9 was tested for cutting activity in plasma in an experimental protocol.

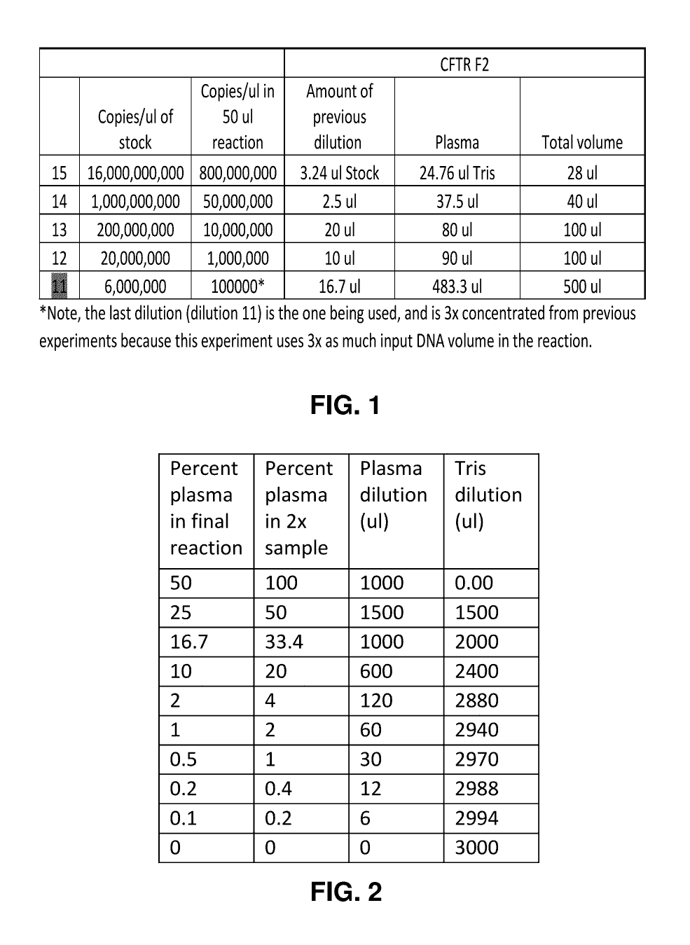

Plasma samples were placed in Streck tubes and in standard tubes. The experiments used an 800 bp amplicon from the cystic fibrosis transmembrane receptor gene. Dilutions were made of CFTR F2 800 bp into plasma with 5 million copies per reaction total (FIG. 1). The percent plasma in reaction after dilution was 50%, 25%, 16.7%, 10%, 2%, 1%, 0.5%, 0.2%, 0.1%, and 0% (FIG. 2).

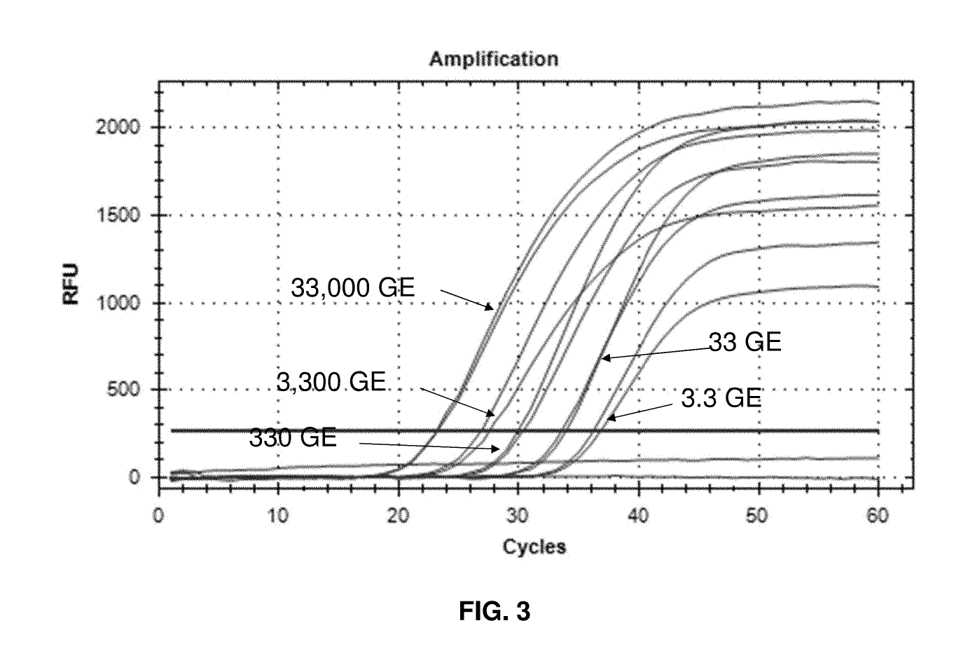

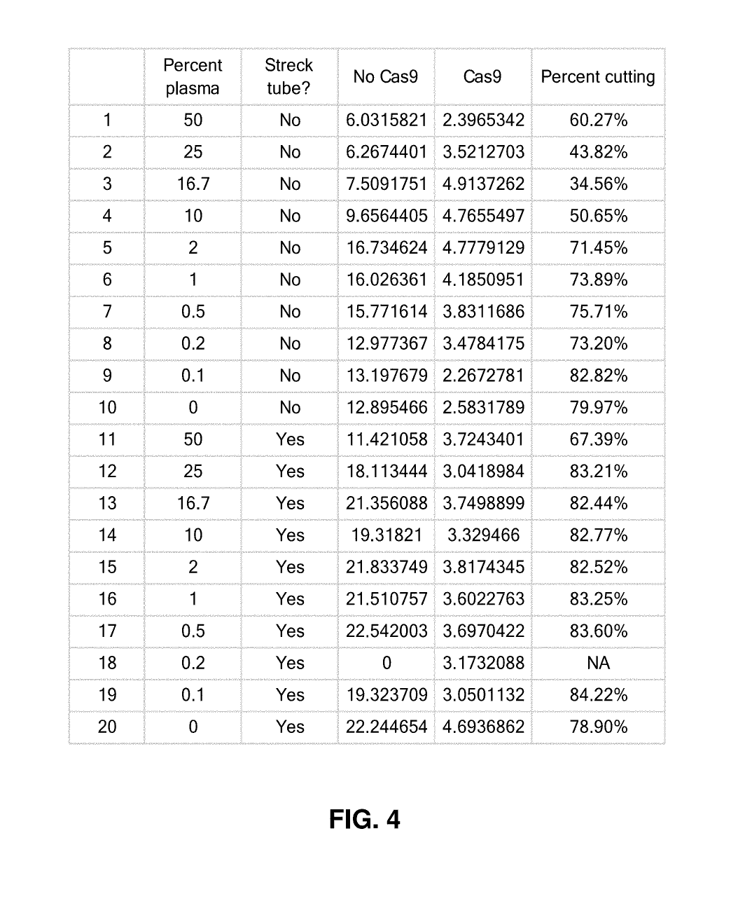

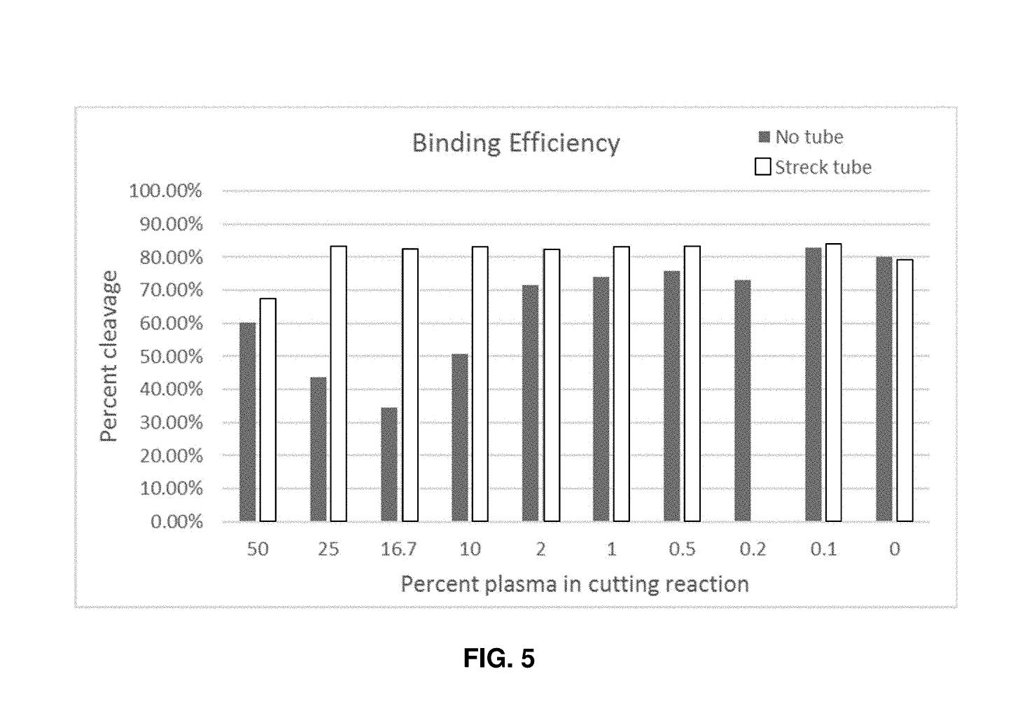

Cas9 with guide RNA was added and allowed to cut. qPCR was then used to probe across the cut site. For qPCR, samples were diluted 1/100, and then 5 ul were used per 20 ul reaction. The qPCR results were analyzed from amplifying, post-cutting, from dilutions (FIGS. 3 and 4). The qPCR results indicated cleavage as a function of plasma amount (FIG. 5). For example, every replicate in a Streck tube demonstrated greater than 60% cutting efficiency by Cas9 in the CFTR amplicon. Cas9 exhibited detectable cutting, even in standard, non-Streck tubes.

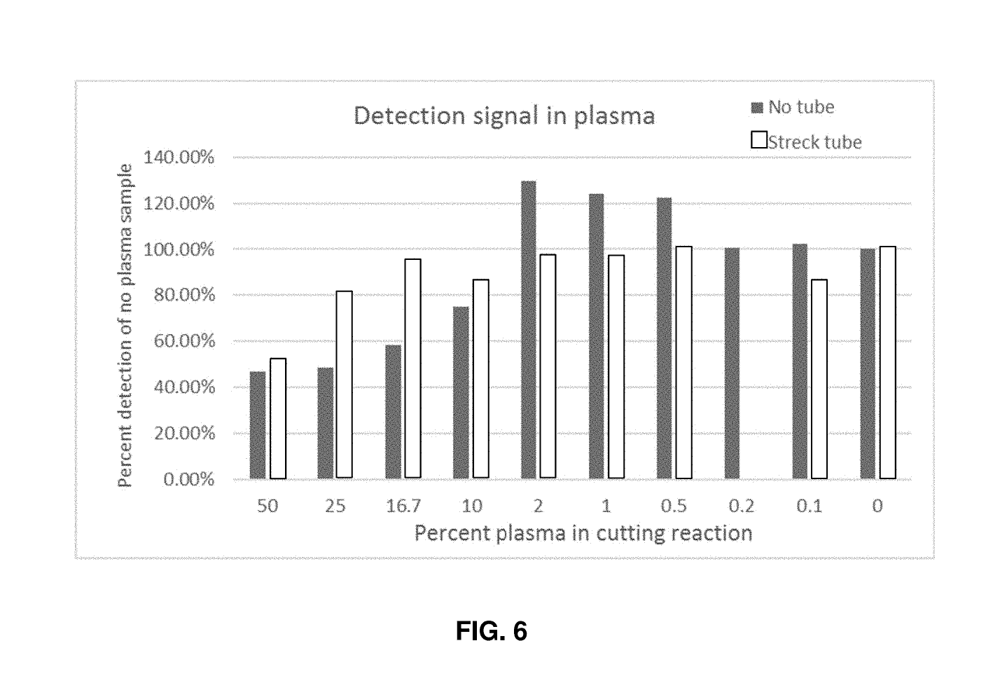

The results also indicated a relationship between the qPCR signal and percent plasma (FIG. 6). For example, the data show Cas9 exhibits detectable cutting in Na-EDTA plasma. For the reactions performed in straight plasma, cutting efficiency in 2% plasma or lower resembled no plasma cutting efficiency (82.82% for in plasma compared to 79.97% in no plasma). For the reactions performed in plasma incubated in a Streck tube, the cutting efficiency in 25% plasma or lower resembled no-plasma cutting efficiency (83.14% compared to 78.90%). Further, there was 60-67% cutting for the 50% plasma samples. In 50% plasma, CRISPR/Cas9 complexes retained 75% activity. Results of the data show that Cas endonuclease and homologs thereof bind to target DNA under guidance of guide RNA in plasma.

In another example, a sample was obtained from a patient having had a tumor. Previous laboratory tests that provided the nucleic acid sequences of driver and passenger mutations associated with the tumor were maintained in a database for future monitoring. After treatment for the patient's cancer was completed and the patient was identified to be in remission, monitoring of the patient's cancer status was initiated. In order to monitor the patient, samples were obtained at subsequent time points. Cas endonuclease/guide RNA complexes specific to the target nucleic acid suspected to contain the passenger mutations of the patient were provided directly into the samples. Detection of the target nucleic acid was performed by methods described herein, and the presence of the passenger mutations in the sample was indicative of the presence of a tumor in the patient. That is, the results indicate that the presence of the passenger mutations in the sample is indicative cancer reoccurrence in the specific patient. Furthermore, such methods can be utilized to also determine treatment efficacy by monitoring the expression levels of the passenger mutations at different time points during treatment.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method for monitoring cancer recurrence in an individual, the method comprising:
obtaining a biological sample from an individual having been treated for cancer;
introducing one or more Cas endonuclease/guide RNA complexes into the sample in which guide RNAs target passenger mutations in a sequence specific manner; and
detecting the presence of passenger mutations in the sample.

2. The method of claim 1, wherein Cas endonuclease of the complex is a catalytically inactive homolog thereof.

3. The method of claim 1, further comprising the step of repeating the obtaining, introducing and detecting steps serially over time.

4. The method of claim 1, further comprising introducing an exonuclease to the sample to digest unbound nucleic acid.

5. The method of claim 1, further comprising quantifying said passenger mutations in order to determine therapeutic efficacy.

6. The method of claim 1, wherein the detecting step comprises hybridizing the bound nucleic acid to a probe or to a primer for detection or amplification, or labeling the nucleic acid with a detectable label.

7. The method of claim 1, wherein the detecting step comprises connecting the bound nucleic acid to a particle or to a column and removing other components of the sample.

8. The method of claim 7, wherein the particle comprises an agent that binds to at least one protein to form a complex.

9. The method of claim 8, wherein the particle comprises magnetic or paramagnetic material and the determining step further comprises applying a magnetic field to separate the complex from the other components of the sample.

10. The method of claim 1, wherein the detecting step comprises applying the sample to a column.

11. The method of claim 10, wherein the bound target nucleic acid is separated from unbound nucleic acid in the sample by size exclusion, ion exchange, or adsorption.

12. The method of claim 11, wherein the detecting step comprises gel electrophoresis.

13. The method of claim 5, further comprising providing a report describing the presence of the passenger mutation of the individual.

14. The method of claim 1, wherein the sample is bile, blood, plasma, serum, sweat, saliva, urine, feces, phlegm, mucus, sputum, tears, cerebrospinal fluid, synovial fluid, pericardial fluid, lymphatic fluid, semen, vaginal secretion, products of lactation or menstruation, amniotic fluid, pleural fluid, rheum, or vomit.

15. The method of claim 1, wherein the sample comprises a liquid biopsy sample and the nucleic acid comprises cell free DNA.

16. The method of claim 5, further comprising identifying a treatment based on the presence of the passenger mutation and providing a report describing the identified treatment.

17. The method of claim 1, further comprising cataloging the nucleic acid sequence of the passenger mutation into a database.
